(19) United States
(12) Patent Application Publication
(10) Pub. No.: US 2016/0070852 A1
(43) Pub. Date: Mar. 10, 2016

(54) PROGNOSIS PREDICTION FOR ACUTE MYELOID LEUKEMIA BY A 3-MICRORNA SCORING METHOD

(71) Applicant: NATIONAL TAIWAN UNIVERSITY, Taipei (TW)

(72) Inventors: Wen-Chien CHOU, Taipei (TW); Hwei-Fang TIEN, Taipei (TW); Eric Y. CHUANG, Taipei (TW); Yu-Chiao CHIU, Taipei (TW); Ming-Kai CHUANG, Taipei (TW)

(21) Appl. No.: 14/477,248

(22) Filed: Sep. 4, 2014

(51) Int. Cl. G06F 19/20; G06F 19/00; C12Q 1/68

(52) U.S. Cl. CPC G06F 19/20; C12Q 1/6886; G06F 19/3431; C12Q 2600/178; C12Q 2600/158

(57) ABSTRACT

The present invention relates to a scoring method for predicting the survival of a de novo AML patient based on the expression level of microRNAs mir-9, mir-155 and mir-203 in the patient. Patients with higher scores are associated with shorter overall survival. This scoring method is simple, powerful, and widely applicable for risk stratification of AML patients.

### BACKGROUND OF THE INVENTION

1. Field of the Invention

The present invention relates to a scoring method comprising 3 microRNAs for predicting the post-treatment survival prospect of an acute myeloid leukemia patient.

2. Description of the Related Art

MicroRNA is a class of small, non-coding RNA which is derived from precursor RNA through processing by protein complex including Dicer and Drosha. It regulates gene expression post-transcriptionally by either degradation of mRNA or inhibition of translation via binding at the 3′-untranslated region of their target genes. The roles of microRNAs in carcinogenesis are very complex.

In acute myeloid leukemia (AML), microRNAs are involved in hematopoietic cell differentiation, proliferation, and survival, and have impact on treatment response and outcome. Different microRNA expression profiles are seen in various cytogenetic groups of AML. Moreover, AMLs with specific gene mutations also harbor distinct sets of microRNA signatures. A particularly important feature of microRNA expression is its role in prognosis. More and more studies have demonstrated both positive and negative roles of microRNAs in gene regulations and their implications on the prognosis or leukemogenesis of AML. Higher expression of a single microRNA, miR-181a, appears to be an independent favorable prognostic factor in cytogenetically normal AML. On the other hand, high expression of individual miR-191, miR-199a, or miR-155, and low expression of miR-212 or miR-29 were reported as poor prognostic factors in AML. We reason that multiple microRNAs are usually involved in specific physiological pathways and may in concert influence the response to chemotherapy in AML patients, so expression levels of multiple microRNAs may be more powerful to predict the prognosis in these patients. As a highly heterogeneous disease, AML needs fine risk stratification to get an optimal outcome of patients. Incorporating expression of multiple relevant microRNAs together and taking into account each microRNA's weight in the survival analysis may provide more integrative information in prognostication.

### SUMMARY OF THE INVENTION

To solve the above problem, the present invention aims to provide a simple and user-friendly method for prognostication in AML patients. By repeated rounds of statistical calculation on the database derived from our patients, we achieved a formula: Risk=0.4908 [expression level of hsa-miR-9-5p]+0.2243 [expression level of hsa-miR-155-5p]−0.7187 [expression level of hsa-miR-203]. The prognostic significance of this formula has been validated in another independent cohort, The Cancer Genome Atlas, from the western countries.

The scoring method consists of: (a) detecting expression level of microRNAs mir 9, mir-155 and mir-203, preferably using MAMMU6 as an endogenous control, in the AML patient; (b) calculating a risk score of the AML patient according to the 3-microRNA scoring formula (detailed below); and (c) determining the prognostic risk category of the patient.

Since the scoring system was designed by z-transformed microRNA expression levels as inputs, cohort mean and cohort standard deviation of each of the three microRNAs are required for that formula. For a practical utilization of this scoring system in other clinical institutes or hospitals without cohort dataset, we provide the calculating formula we used in the NTUH dataset:

Risk=0.4908 (−ΔCₜ_{^{hsa-miR-9-5p}}+15.71)/3.60+0.2243 (−ΔCₜ_{^{hsa-miR-155-5p}}±6.94)/1.45−0.7187 (−ΔCₜ_{^{hsa-miR-203}}+17.16)/2.66. Here the ΔCₜ values are Cₜ of the microRNA subtract Cₜ of the endogenous control, preferably, MAMMU6; 15.71 and 3.60 are the mean and standard deviation of ΔCₜ_{^{hsa-miR-9-5p}}. The same annotation applies to hsa-miR-155-5p and hsa-miR-203. For each newly diagnosed patient, a 4-well real-time PCR microRNA assay (probing hsa-miR-9-5p, hsa-miR-155-5p, hsa-miR-203, and MAMMU6) is sufficient to get a prognostic score, which will then be compared with our cohort median score 0.0031 to stratify the risk group. A risk score equal to or lower than 0.0031 indicates that the patient has a favorable prospect of post-treatment survival.

In one embodiment of the present invention, the AML patient is de novo AML patient.

Another aspect of the present invention is to provide a kit for detecting the expression of microRNAs, wherein the kit comprises oligonucleotides capable of detecting the expression of microRNAs mir-9, mir-155 and mir-203.

Preferably, the kit further comprises oligonucleotides capable of detecting an endogenous control. In a preferable embodiment, the endogenous control is MAMMU6.

### BRIEF DESCRIPTION OF THE DRAWINGS

Non-limiting and non-exhaustive embodiments will be described in conjunction with the accompanying drawings. Understanding that these drawings depict only several embodiments in accordance with the disclosure and are, therefore, not to be intended to limit its scope, the disclosure will be described with specificity and detail through use of the accompanying drawings, in which:

FIG. 1 shows the flow chart of microRNA analysis of the embodiment of the present invention.

FIG. 2(a) shows the distribution of risk scores among the 138 patients in the NTHU group. FIG. 2(b) shows the distribution of risk scores among TCGA cohort.

FIG. 3(a) shows the OS comparison of AML patients with lower score and those with higher score among NTHU group. FIG. 3(b) shows the OS comparison of AML patients with lower score and those with higher score among TCHA cohort. FIG. 3(c) shows the OS comparison of AML patients with a normal karyotype with lower scores and those with higher scores among the NTHU group. FIG. 3(d) is a scatter gram shows higher scores are associated with lower probability of getting complete remission (CR).

### DETAILED DESCRIPTION OF THE PREFERRED EMBODIMENTS

Hereinafter, illustrative embodiments and examples of the present disclosure will be described in detail with reference to the accompanying drawings so that inventive concept may be readily implemented by those skilled in the art.

However, it is to be noted that the present disclosure is not limited to the illustrative embodiments but can be realized in various other ways. In the drawings, certain parts not directly relevant to the description are omitted to enhance the clarity of the drawings, and like reference numerals denote like parts throughout the whole document.

Throughout the whole document, the term “comprises or includes” and/or “comprising or including” used in the document means that one or more other components, steps, operations, and/or the existence or addition of elements are not excluded in addition to the described components, steps, operations and/or elements. The terms “about or approximately” or “substantially” are intended to have meanings close to numerical values or ranges specified with an allowable error and intended to prevent accurate or absolute numerical values disclosed for understanding of the present invention from being illegally or unfairly used by any unconscionable third party.

This application provides a method for predicting a clinical outcome (e.g., the post-treatment survival) of an AML patient based on the expression patterns of one or more microRNAs that are associated with the clinical outcome.

The utilization of the scoring method is described as follows. A group of AML patients are recruited. The expression levels of a number of microRNAs in bone marrow cells are determined by methods known in the art, e.g., real-time PCR or micro-array analysis. The expression level of each microRNA thus determined is normalized by the expression level of an internal control, such as a MAMMU6, in the same patient to obtain a normalized expression levels for the three component microRNAs.

### Example

Hereinafter, the present disclosure will be specifically described with reference to examples and drawings. However, the present disclosure is not limited to the examples and the drawings.

[Material and Method]

(a) Patents

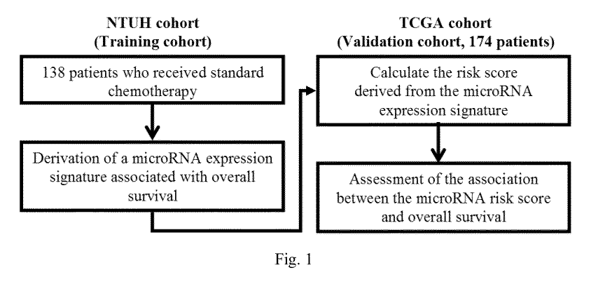

A total of 195 consecutive adult patients (> or =15 years of age) with newly diagnosed de novo AML from 1995 to 2007 at the National Taiwan University Hospital (NTUH) who had adequate cryopreserved bone marrow cells for microRNA analysis were recruited. Patients with antecedent hematological diseases or therapy-related AML were excluded. This experiment was performed in accordance with the Declaration of Helsinki and was approved by the Institutional Review Board of the NTUH. Among these primary AML patients, 138 (70.7%) received standard intensive chemotherapy. The remaining 57 patients received palliative care or low-dose chemotherapy due to poor performance status or per patients' wish. All the 195 patients were included for correlation analysis between expression of specific microRNA and other parameters, but only the 138 patients who received standard intensive chemotherapy were included for survival analysis. AML cohort from TCGA (The Cancer Genome Atlas) was used, which contains publically available data of microRNA and clinical information, as a validation cohort (FIG. 1).

(b) Quantification of microRNA Expression

Mononuclear cells were isolated from bone marrow samples obtained at diagnoses, followed by cryopreservation. RNA was extracted by TriZol method. One μg RNA was subject to TaqMan MicroRNA Reverse Transcription kit (Applied Biosystems) and the microRNA profiling was assayed by TaqMan Array Human microRNA A card (Applied Biosystems) on 7900HT real time PCR machine. The amplification curves were converted into numeric tables using Applied Biosystems SDS2.3 software. MicroRNAs that cannot be detected by 40 cycles (C_{T}>40) were marked as undetermined.

(c) Establishment of a Risk Scoring Method

To build a risk scoring method based on microRNA expression levels, the association between overall survival (OS) and the expression levels of individual microRNAs was first analyzed. Here the prognostic significance of each microRNA expression on survival was measured using univariate Cox proportional hazards regression model. The expression of microRNAs with top significance on survival (univariate Cox P<0.005) was then applied to the multivariate Cox model in order to find those microRNAs whose expression could independently predict survival. The expression of microRNAs with significant association with OS from the multivariate tests (multivariate Cox P<0.1) was selected to generate the risk scoring method, in which the expression of component microRNAs went through another round of multivariate Cox regression test to get beta values as weights. The expression of microRNAs with higher prognostic significance on survival was weighted more. The microRNA-based risk scoring method is defined as

$${Risk}_{p} = {Σ}_{{miRNA}_{i} + {componentsmiRNA}_{i}} {Beta}_{i} \cdot {miRNA}_{i} ( p ) ,$$

where P denotes the patient accession number, Betaᵢ means the weight of the microRNA probe i, and miRNAi represents the log-transformed expression level of microRNA probe i. The sum of Betaᵢ ΣmiRNA p of all the component microRNAs is the estimated risk scores for Patient P.

A ten-thousand-time random permutation test was performed to ensure the performance of our scoring method. For each iteration of the random permutation, the same number of microRNAs were randomly selected from the microRNA dataset into construction of a “random scoring method”, where appropriate weights were assigned according to the procedures discussed above. Each random scoring method was tested for prognostic significance using the univariate Cox model. After 10,000 iterations, the empirical P value of the proposed risk system could be calculated as the fraction of random scoring methods that achieved better univariate Cox P values than the proposed risk system. The smaller the empirical P value is, the better the proposed risk scoring method outperforms random microRNA combinations.

(d) Statistical Analysis

OS was measured from the date of first diagnosis to death from any cause or the last follow-up. To exclude any confounding influences from allogeneic hematopoietic stem cell transplantation (HSCT), patients who received this procedure were censored on the day of cell infusion. Kaplan-Meier estimation was adopted to plot survival curves and used log rank tests to examine the difference between groups. The whole patient population was included for analyses of the correlation between microRNA expression-based risk scoring method and clinical characteristics; however, only those receiving standard intensive chemotherapy were included in analyses of survivals. All statistical analyses were accomplished with XLSTAT statistical analysis software edition 2010.5.02 (Addinsoft, Deutschland, Germany).

[Results]

(a) Constriction of the 3-microRNA Risk Scoring Method

To define a microRNA scoring method predictive of the risk in AML patients, screening single microRNA expression that was individually associated with OS in the NTUH cohort (as a discovery dataset, n=138) was performed. The univariate Cox proportional regression model identified expression of eleven microRNAs with significant association with OS (P<0.005), including hsa-miR-9-5p, hsa-miR-146a, hsa-miR-222, hsa-miR-128, hsa-miR-181a, hsa-miR-125b, hsa-miR-196b, hsa-miR-155-5p, hsa-miR-224, hsa-miR-203, and hsa-miR-339-3p (ranked by increasing Cox P values). To further pinpoint microRNAs expression with independent power of survival prediction, we introduced the expression of eleven microRNAs into a multivariate Cox model and identified high expression of hsa-miR-9-5p (Accession number: MIMAT0000441) and hsa-miR-155-5p (MIMAT0000646) were independently associated with poor OS, while that of hsa-miR-203 (MIMAT0000264) had a trend of association with favorable OS, with multivariate Cox P=0.005, 0.033, and 0.080, respectively. By focusing these 3 microRNAs, a risk scoring method was constructed as follows:

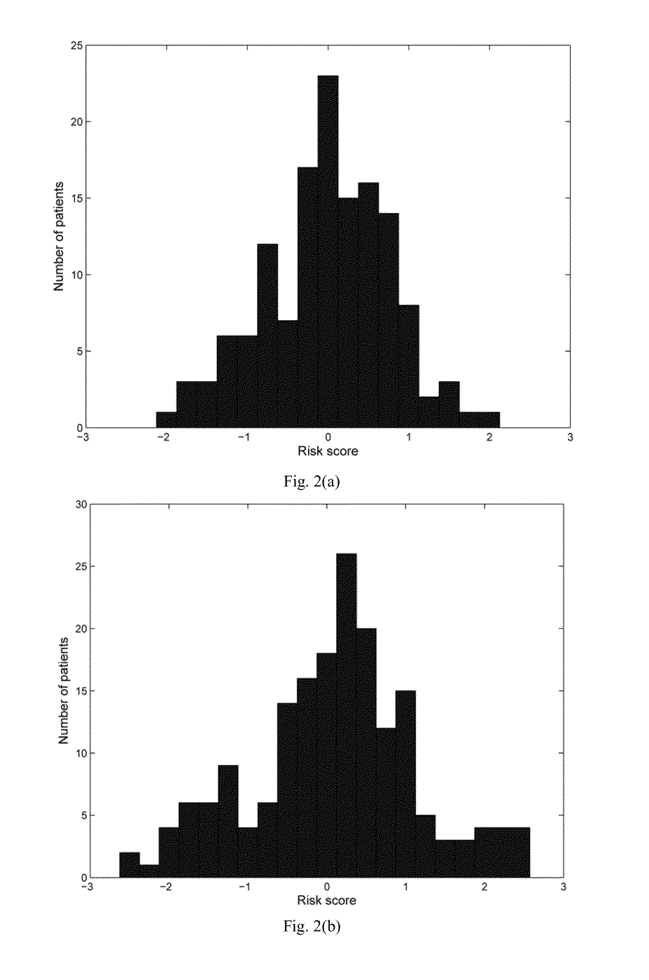

Risk=0.4908 [hsa-miR-9-5p]+0.2243 [hsa-miR-155-5p]−0.7187 [hsa-miR-203], where the weights of microRNAs are beta values from multivariate Cox analysis and the expression levels of microRNAs are z-transformed (ie. subtracting the mean and then divided by the standard deviation) across patients so that each microRNA has zero mean and unit standard deviation. The distribution of risk scores among the 138 patients in the NTUH discovery group was approximately normally shaped and ranged from −2.01 to 1.97, with median, mean and standard deviation of 0.01, 2.87e-15 and 0.78, respectively (FIG. 2(a)). The same situation could also apply to TCGA cohort (FIG. 2 (b)). The risk score served as a good survival predictor in our dataset (univariate Cox P=1.41×10⁻⁷, Log-rank P=1.03×10⁻⁵). The scoring method outperformed almost all the ten thousand random selections (empirical P=6×10⁻⁴), suggestive of the high performance and non-randomness of our proposed system.

(b) Correlation of Clinical and Molecular Characteristics with Scoring Method

Higher scores were positively associated with older age, higher counts of white blood cells, platelets, and blasts, but mutually exclusive with favorable cytogenetics (see Table 1).

TABLE 1
Correlation between microRNA score and clinical data, FAB subtypes and
chromosomal abnormalities in AML patients (n = 195)

| | | | microRNA Score | | | | |
|---|---|---|---|---|---|---|---|
| Variant | Total | | Low (n = 97) | | High (n = 98) | | P |
| Median age, y (range) | 54 | (15-89) | 50 | (18-89) | 61.5 | (15-88) | 0.210 |
| Age, in groups | | | | | | | |
| >60 | 80 | (41.0%) | 30 | (30.9%) | 50 | (51.0%) | 0.006 |
| >50 | 108 | (56.8%) | 48 | (49.5%) | 60 | (61.2%) | 0.144 |
| Gender | | | | | | | |
| male | 110 | (56.4%) | 57 | (58.8%) | 53 | (54.1%) | 0.564 |
| Lab data | | | | | | | |
| WBC (×10³/μL) | 23.54 | (0.38-423.0) | 15.37 | (0.38-189.1) | 29.1 | (0.65-423.0) | <0.001 |
| Blasts (×10³/μL) | 12.03 | (0-369.0) | 5.95 | (0-149.8) | 16.45 | (0-369.0) | <0.001 |
| Hemoglobin, g/dL | 7.8 | (3.3-16.2) | 7.6 | (3.3-13.1) | 8.1 | (4.1-16.2) | 0.212 |
| Platelets (×10³/μL) | 41.0 | (2-455) | 33.0 | (2-226) | 47.0 | (7-455) | 0.018 |
| LDH (U/L) | 875.0 | (271-8116) | 867.0 | (271-6885) | 889.0 | (274-8116) | 0.587 |
| FAB | | | | | | | 0.007 |
| M0 | 2 | (1.0%) | 1 | (1.0) | 1 | (1.0%) | >0.999 |
| M1 | 37 | (19.0%) | 18 | (18.6%) | 19 | (19.4%) | >0.999 |
| M2 | 59 | (30.3%) | 34 | (35.1%) | 25 | (25.5%) | 0.163 |
| M3 | 17 | (8.7%) | 15 | (15.5%) | 2 | (2.0%) | 0.001 |
| M4 | 63 | (32.3%) | 23 | (23.7%) | 30 | (40.8%) | 0.014 |
| M5 | 11 | (5.6%) | 3 | (3.1%) | 8 | (8.2%) | 0.213 |
| Karyotype SWOG* | N = 181 | | N = 94 | | N = 87 | | <0.001 |
| Favorable | 39 | (21.5%) | 32 | (34.0%) | 7 | (8.0%) | <0.001 |
| Intermediate | 124 | (68.5%) | 52 | (55.3%) | 72 | (82.8%) | 0.005 |
| Unfavorable | 18 | (9.9%) | 10 | (10.6%) | 8 | (9.2%) | 0.630 |
| Normal | 92 | (50.3%) | 40 | (42.6%) | 52 | (58.4%) | 0.039 |
| Isolated +8 | 8 | (4.4%) | 2 | (2.1%) | 6 | (6.9%) | 0.156 |

*Southwest oncology group cytogenetic risk category: favorable: inv(16)/t(16; 16)/del(16q), t(15; 17) with/without secondary aberrations; t(8; 21) lacking del(9q) or complex karyotypes; intermediate: Normal, +8, +6, −Y, del(12p); unfavorable: del(5q)/−5, −7/del(7q), abn 3q, 9q, 11q, 20q, 21q, 17p, t(6; 9), t(9; 22) and complex karyotypes (≧3 unrelated abn)

Genetic mutation profiles were also different between the high and low score groups: patients with higher scores more often had NPM1 mutation, FLT3-ITD and MLL-PTD, but less likely had CEBPA mutation (Table 2).

TABLE 2
Correlation of microRNA score with other gene alterations

| | | MicroRNA Score | | |
|---|---|---|---|---|
| | Total | Low | High | |
| Mutation | (n = 189) | (n = 96) | (n = 93) | P |
| NPM1 | 46 (24.3%) | 17 (17.7%) | 29 (31.2%) | 0.041 |
| FLT3-ITD | 48 (25.4%) | 12 (12.5%) | 36 (38.7%) | <0.001 |
| NPM1⁺/FLT3-ITD⁻ | 25 (13.2%) | 10 (10.4%) | 15 (16.1%) | 0.287 |
| CEBPA^{double} | 13 (6.9%) | 10 (10.4%) | 3 (3.2%) | 0.082 |
| CEBPA | 19 (10.1%) | 15 (15.6%) | 4 (4.3%) | 0.014 |
| WT1 | 11 (5.8%) | 3 (3.1%) | 8 (8.6%) | 0.129 |
| RUNX1 | 26 (13.8%) | 9 (9.4%) | 17 (18.3%) | 0.092 |
| IDH1 | 11 (5.8%) | 6 (6.3%) | 5 (5.4%) | >0.999 |
| IDH2 | 25 (13.2%) | 10 (10.4%) | 15 (16.1%) | 0.287 |
| FLT3-TKD | 18 (9.5%) | 9 (9.4%) | 9 (9.7%) | >0.999 |
| MLL-PTD | 9 (4.8%) | 1 (1.0%) | 8 (8.6%) | 0.017 |
| KIT | 8 (4.2%) | 5 (5.2%) | 3 (3.2%) | 0.721 |
| KRAS | 7 (3.7%) | 5 (5.2%) | 2 (2.2%) | 0.445 |
| NRAS | 28 (14.8%) | 11 (11.5%) | 17 (18.3%) | 0.222 |
| ASXL1 | 21 (11.1%) | 14 (14.6%) | 7 (7.5%) | 0.165 |
| TET2 | 30 (15.9%) | 15 (15.6%) | 15 (16.1%) | >0.999 |
| DNMT3A | 33 (17.5%) | 12 (12.5%) | 21 (22.6%) | 0.086 |

(c) Survival Analysis

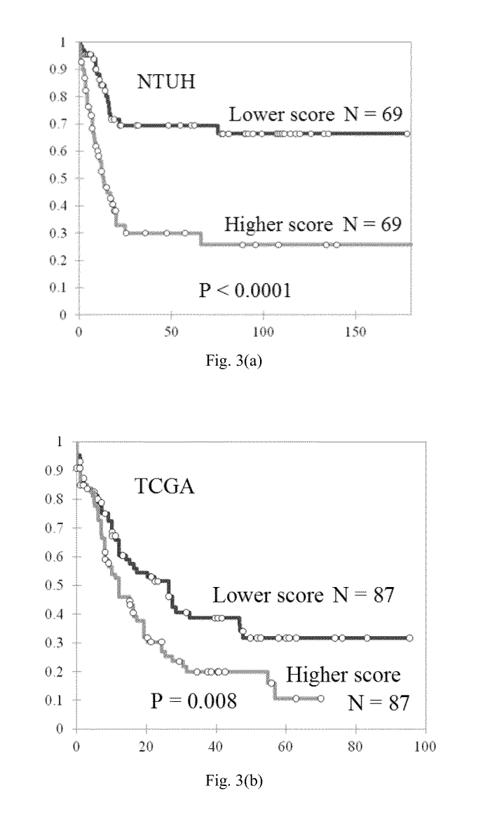

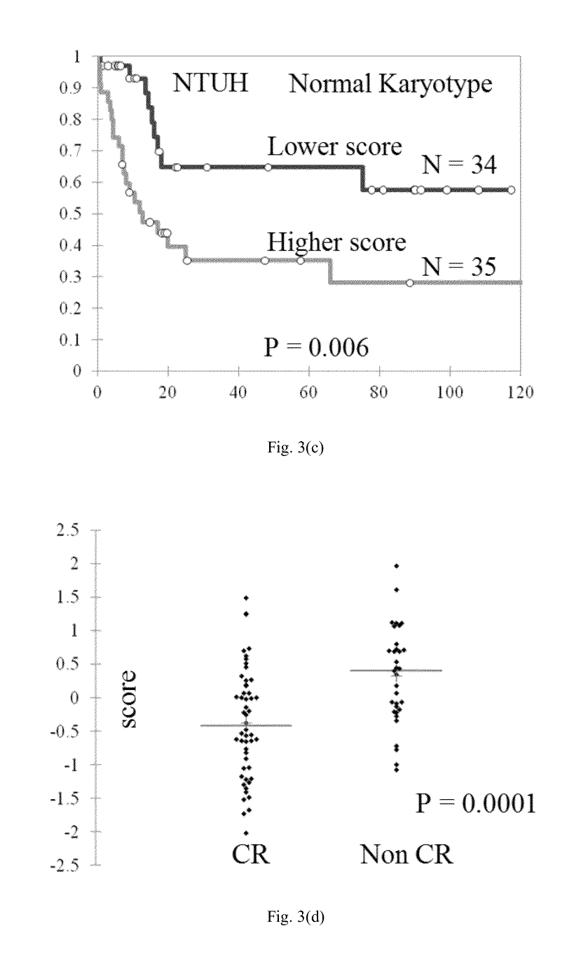

AML patients with higher scores had significantly shorter OS compared with those with lower scores (median 13.5 months vs. not reached, P<0.0001, FIG. 3(a)). The prognostic significance of the scoring method was validated in TCGA AML cohort (median 12.2 vs 26.4 months, P=0.008, FIG. 3(b)), which is the only publically available AML cohort with survival and microRNA expression data. When we restricted the analysis in our patients with a normal karyotype, the OS of patients with higher scores still fared worse (median 17.0 months vs. not reached, P=0.006, FIG. 3(c)). The patients with lower scores were more likely to achieve complete remission (CR) after induction chemotherapy than those with higher scores (P=0.0001, FIG. 3(d)).

(d) Multivariate Analysis

Because high scores seemed to be associated with other poor prognostic variables (Tables 1 and 2), we sought to investigate whether the score in the scoring method functioned as an independent factor. We included several well-known prognostic factors as co-variables and high score appeared to be a highly independent risk factor. Notably, the independency of our scoring method still held true in TCGA cohort (Table 3). Further analysis by integrating the co-variables, we noted the patients with more poor prognostic factors had shorter OS in both ours and TCGA patients.

TABLE 3
Multivariate analysis (Cox regression) for the
overall survival in NTUH and TCGA AML cohorts

| | Hazard ratio | | 95% confidence interval | | P value | |
|---|---|---|---|---|---|---|
| Variables | NTUH | TCGA | NTUH | TCGA | NTUH | TCGA |
| Age* | 2.350 | 2.025 | 1.319~4.187 | 1.272~3.226 | 0.004 | 0.003 |
| WBC† | 2.189 | 0.864 | 1.118~4.284 | 0.550~1.355 | 0.022 | 0.524 |
| Karyotype‡ | 1.388 | 1.482 | 0.766~2.513 | 1.072~2.048 | 0.279 | 0.017 |
| NPM1/FLT3-ITD\|\| | 0.118 | 0.823 | 0.016~0.885 | 0.460~1.472 | 0.038 | 0.512 |
| CEBPA^{double}¶ | 0.431 | — | 0.179~1.037 | — | 0.060 | — |
| miRNA score | 2.079 | 1.544 | 1.407~3.073 | 1.229~1.940 | <0.001 | <0.001 |

*Age older than 50 y relative to age 50 y or younger.
†WBC greater than 50 000/μL vs less than or equal to 50 000/μL.
‡Unfavorable cytogenetics versus others.
||NPM1⁺/FLT3-ITD⁻ vs other subtypes.
¶CEBPA double-mutation vs others.

(e) Comparison of Prognostic Significance Between the Scoring Method and Single microRNA Expression

To see if the scoring method was more powerful than single microRNA expression in predicting prognosis, we added the expression of individual component microRNAs, hsa-miR-9-5p, hsa-miR-155-5p, and hsa-miR-203, and hsa-miR-181a, a microRNA whose up-regulation is highly associated with favorable prognosis in AML, into the multivariate analysis in addition to the co-variables shown in Table 3. The results showed that the 3-microRNA signature outperformed all the single microRNA expression (Tables 4-7).

TABLE 4
Multivariate analysis (Cox regression) for the overall survival in NTUH and
TCGA AML cohorts, including both microRNA score and miR-181a expression

| | Hazard ratio | | 95% confidence interval | | P value | |
|---|---|---|---|---|---|---|
| Variables | NTUH | TCGA | NTUH | TCGA | NTUH | TCGA |
| Age* | 1.855 | 2.226 | 1.030~3.344 | 1.379~3.594 | 0.039 | 0.001 |
| WBC† | 1.658 | 1.064 | 0.937~2.934 | 0.657~1.722 | 0.082 | 0.801 |
| Karyotype‡ | 1.969 | 1.696 | 0.915~4.235 | 1.009~2.853 | 0.083 | 0.046 |
| NPM1/FLT3-ITD\|\| | 0.572 | 0.933 | 0.240~1.361 | 0.523~1.663 | 0.206 | 0.813 |
| CEBPA^{double}¶ | 0.178 | — | 0.023~1.351 | — | 0.095 | — |
| microRNA score | 2.277 | 1.358 | 1.566~3.310 | 1.088~1.695 | <0.0001 | 0.007 |
| miR-181a | 0.777 | 0.824 | 0.607~0.995 | 0.660~1.029 | 0.046 | 0.088 |

*Age older than 50 y relative to age 50 y or younger.
†WBC greater than 50 000/μL vs less than or equal to 50 000/μL.
‡Unfavorable cytogenetics versus others.
||NPM1⁺/FLT3-ITD⁻ vs other subtypes.
¶CEBPA double-mutation vs others.

TABLE 5
Multivariate analysis (Cox regression) for the overall survival in NTUH
and TCGA AML cohorts, including both microRNA score and miR-9 expression

| | Hazard ratio | | 95% confidence interval | | P value | |
|---|---|---|---|---|---|---|
| Variables | NTUH | TCGA | NTUH | TCGA | NTUH | TCGA |
| Age* | 1.938 | 2.434 | 1.043~3.597 | 1.503~3.940 | 0.037 | 0.0001 |
| WBC† | 1.904 | 1.082 | 1.072~3.381 | 0.666~1.758 | 0.028 | 0.751 |
| Karyotype‡ | 2.217 | 1.872 | 1.001~4.907 | 1.128~3.108 | 0.050 | 0.015 |
| NPM1/FLT3-ITD\|\| | 0.598 | 1.004 | 0.249~1.436 | 0.557~1.808 | 0.250 | 0.991 |
| CEBPA^{double}¶ | 0.234 | — | 0.031~1.799 | — | 0.163 | — |
| microRNA score | 2.579 | 1.525 | 1.382~4.813 | 1.172~1.984 | 0.003 | 0.002 |
| miR-9 | 0.818 | 0.937 | 0.510~1.314 | 0.737~1.191 | 0.406 | 0.594 |

*Age older than 50 y relative to age 50 y or younger.
†WBC greater than 50 000/μL vs less than or equal to 50 000/μL.
‡Unfavorable cytogenetics versus others.
||NPM1⁺/FLT3-ITD⁻ vs other subtypes.
¶CEBPA double-mutation vs others.

TABLE 6
Multivariate analysis (Cox regression) for the overall survival in NTUH and
TCGA AML cohorts, including both microRNA score and miR-203 expression

| | Hazard ratio | | 95% confidence interval | | P value | |
|---|---|---|---|---|---|---|
| Variables | NTUH | TCGA | NTUH | TCGA | NTUH | TCGA |
| Age* | 1.538 | 2.360 | 0.595~3.984 | 1.459~3.818 | 0.374 | 0.0001 |
| WBC† | 1.399 | 1.118 | 0.558~3.507 | 0.690~1.811 | 0.474 | 0.652 |
| Karyotype‡ | 2.186 | 1.865 | 0.526~9.082 | 1.124~3.095 | 0.282 | 0.016 |
| NPM1/FLT3-ITD\|\| | 1.839 | 0.972 | 0.490~6.906 | 0.544~1.735 | 0.367 | 0.923 |
| CEBPA^{double}¶ | 0.489 | — | 0.044~5.470 | — | 0.561 | — |
| microRNA score | 3.730 | 1.574 | 1.515~9.183 | 1.075~2.305 | 0.004 | 0.020 |
| miR-203 | 1.376 | 1.097 | 0.602~3.146 | 0.739~1.628 | 0.449 | 0.646 |

*Age older than 50 y relative to age 50 y or younger.
†WBC greater than 50 000/μL vs less than or equal to 50 000/μL.
‡Unfavorable cytogenetics versus others.
||NPM1⁺/FLT3-ITD⁻ vs other subtypes.
¶CEBPA double-mutation vs other subtypes.

TABLE 7
Multivariate analysis (Cox regression) for the overall survival in NTUH and
TCGA AML cohorts, including both microRNA score and miR-155 expression

| | Hazard ratio | | 95% confidence interval | | P value | |
|---|---|---|---|---|---|---|
| Variables | NTUH | TCGA | NTUH | TCGA | NTUH | TCGA |
| Age* | 0.551 | 2.424 | 0.306~0.992 | 1.497~3.925 | 0.047 | 0.0001 |
| WBC† | 1.954 | 1.014 | 1.122~3.403 | 0.623~1.648 | 0.018 | 0.957 |
| Karyotype‡ | 2.050 | 1.887 | 0.944~4.448 | 1.138~3.129 | 0.069 | 0.014 |
| NPM1/FLT3-ITD\|\| | 0.545 | 1.169 | 0.228~1.302 | 0.632~2.161 | 0.172 | 0.620 |
| CEBPA^{double}¶ | 0.135 | — | 0.018~1.007 | — | 0.051 | — |
| microRNA score | 2.328 | 1.333 | 1.498~3.617 | 1.061~1.676 | 0.0001 | 0.014 |
| miR-155 | 0.901 | 1.279 | 0.646~1.256 | 1.007~1.624 | 0.537 | 0.043 |

*Age older than 50 y relative to age 50 y or younger.
†WBC greater than 50 000/μL vs less than or equal to 50 000/μL.
‡Unfavorable cytogenetics versus others.
||NPM1⁺/FLT3-ITD⁻ vs other subtypes.
¶CEBPA double-mutation vs other subtypes.

(f) Clinically Practical Scoring Method Using Real-Time PCR microRNA Assay

Since the scoring method was designed by z-transformed microRNA expression levels as inputs, cohort mean and cohort standard deviation of each of the three microRNAs are required for that formula. For a practical utilization of this scoring method in other clinical institutes or hospitals without cohort dataset, we provide the calculating formula we used in the NTUH dataset:

Risk=0.4908(−ΔCt_{hsa-miR-9-5p}+15.71)/3.60+0.2243(−ΔCt_{hsa-miR-155-5p}+6.94)/1.45−0.7187(−ΔCt_{hsa-miR-203}+17.16)/2.66.

Here the Δ Ct values are Ct of the microRNA subtract Ct of the endogenous control MAMMU6; 15.71 and 3.60 are the mean and standard deviation of Δ Ct_{hsa-miR-9-5p}. The same annotation applies to hsa-miR-155-5p and hsa-miR-203. For each newly diagnosed patient, a 4-well real-time PCR microRNA assay (probing hsa-miR-9-5p, hsa-miR-155-5p, hsa-miR-203, and MAMMU6) is sufficient to get a prognostic score, which will then be compared with our cohort median score 0.0031 to stratify the risk group.

In the present invention, we took advantage of an integration of comprehensive clinical, genetic, and array data of our cohort to reach a simple but powerful 3-microRNA signature for prediction of clinical outcome, considering the expression levels and weights of only 3 microRNAs, which were sieved through repeated rounds of statistic calculation to ensure the strong and independent influence on prognosis. The power of this score was validated by TCGA cohort, an independent validation set of patients who were studied by a different microRNA quantification platform. Thus, the scoring method of the present invention is independent of both patient populations and quantification methods. Although both NTHU and TCGA patients were not prospective cohorts, the very high significance of the 3-microRNA signature in prognosis prediction in these two independent populations suggests its high reliability for application in risk stratification. Notably, the integrated 3-microRNA scoring method of the present invention outperformed the expression of individual microRNA, hsa-miR-9-5p, hsa-miR-155-5p, and hsa-miR-203, all of which were component microRNAs in the scoring method, and hsa-miR-181a, whose up-regulation was shown to be a highly favorable prognostic factor in AML. While low scores of the scoring method of the present invention were associated with favorable prognostic factors such as good-risk cytogenetics and gene mutations, multivariate analyses in our cohort and that of TCGA confirmed the independence of this 3-microRNA signature to other important prognosis parameters.

For a practical point of view, the means and standard deviations was applied into the scoring method so that every newly diagnosed AML patient's treatment outcome can be predicted by simple qPCR-based experimental procedures even the labs do not have means or standard deviations of their cohort. All the materials are commercially available and the procedures are fast and can fit into a high-throughput manner.

MiR-155, miR-9, and miR-203 are three core components of the scoring method of the present invention. For hematopoietic cancers, miR-155 has been shown to act as an oncogene and confer poor prognosis, but has been shown contrary in another study. The pathways mediating the functions of miR-155 are very complex, but the biological consequences are largely promotion of cell proliferation, cell cycle progression, and invasion/metastasis. In our cohort, miR-155 is an independent unfavorable prognostic factor, compatible with the previous report. MiR-9 has been shown to inhibit tumorigenicity of cancers, but this molecule can promote metastasis of solid cancers, too. More complicated, its increased expression was shown to be a favorable prognostic factor in medulloblastoma in one report, but a poor prognostic factor in glioma in another study. For AML, miR-9 is the most specifically up-regulated microRNA in MLL-rearranged AML compared with other types of AML, as it is a target of MLL fusion proteins, and its expression directly correlates with disease progression. In our study, miR-9 was an unfavorable prognostic factor. There are less studies about miR-203. This molecule functions as a suppressor of skin stemness by regulating the transition between proliferative basal progenitors and terminally differentiating suprabasal cells in the skin. Its prognostic significance in AML has not been clarified, but it can target ABL1 and suppress BCR-ABL1 expression in chronic myeloid leukemia or some acute lymphoblastic leukemia. In other cancers it usually acts as a tumor suppressor, but upregulation of miR-203 in ovarian cancers is correlated with tumor progression and poor prognosis. In the present invention, miR-203 was found to be a favorable prognostic factor.

In conclusion, the present invention presents a simple and user-friendly 3-microRNA signature as a powerful prognostic factor for AML through multiple rounds of statistical analyses on our cohort and further validation by another independent patient group. This scoring method outperforms the expression of single microRNA in multivariate analysis. Paired microRNA-mRNA analyses suggest association between this signature and the common cancer-related molecular pathways.

While example embodiments have been disclosed herein, it should be understood that other variations may be possible. Such variations are not to be regarded as a departure from the spirit and scope of example embodiments of the present application, and all such modifications as would be obvious to one skilled in the art are intended to be included within the scope of the following claims.

What is claimed is:

1. A scoring method of predicting post-treatment survival of an AML patient, comprising:
(a) detecting expression level of microRNAs mir-9, mir-155 and mir-203, in the AML patient;
(b) calculating a risk score of the AML patient according to a 3-microRNA scoring formula; and
(c) determining the prognostic risk category of the patient.

2. The scoring method of claim 1, wherein the risk score is calculated as follows:
Risk=0.4908[expression level of hsa-miR-9-5p]+0.2243[expression level of hsa-miR-155-5p]−0.7187[expression level of hsa-miR-203].

3. The scoring method of claim 2, wherein a risk score equal to or lower than 0.01 indicates that the patient has a favorable prospect of post-treatment survival.

4. The scoring method of claim 1, wherein the risk score is calculated as follows:
Risk=0.4908 (−ΔCₜ_{^{hsa-miR-9-5p}}±15.71)/3.60+0.2243 (−ΔCₜ_{^{hsa-miR-155-5p}}+6.94)/1.45−0.7187 (−ΔCₜ_{^{hsa-miR-203}}+17.16)/2.66, wherein each ΔCₜ values is Cₜ of the microRNA subtract Cₜ of an endogenous control.

5. The scoring method of claim 4, wherein the endogenous control is MAMMU6.

6. The scoring method of claim 5, wherein a risk score equal to or lower than 0.0031 indicates that the patient has a favorable prospect of post-treatment survival.

7. The scoring method of claim 1, wherein the acute myeloid leukemia patient is de novo AML patient.

8. A kit for detecting the expression of microRNAs, wherein the kit comprises oligonucleotides capable of detecting the expression of microRNAs mir-9, mir-155 and mir-203

9. The kit of claim 8, further comprising oligonucleotides capable of detecting an endogenous control.

10. The kit of claim 9, wherein the endogenous control is MAMMU6.
